(19) United States
(12) Patent Application Publication
(10) Pub. No.: US 2005/0159784 A1
(43) Pub. Date: Jul. 21, 2005

(54) Medical cart

(75) Inventor: Radmond Vincent Arceta, Manassas, VA (US)

Correspondence Address: FOLEY AND LARDNER;SUITE 500, 3000 K STREET NW, WASHINGTON, DC, 20007, US

(73) Assignee: RUBBERMAID COMMERCIAL PRODUCTS LLC

(21) Appl. No.: 11/038,488

(22) Filed: Jan. 21, 2005

Related U.S. Application Data
(60) Provisional application No. 60/537,509, filed on Jan. 21, 2004.
(60) Provisional application No. 60/621,662, filed on Oct. 26, 2004.

(51) Int. Cl. A61N 1/18; A61N 1/20; A61N 1/22; A61N 1/24; A61N 1/26; A61N 1/28; A61N 1/30; A61N 1/32; A61N 1/34; A61N 1/36; A61N 1/362; A61N 1/365; A61N 1/368; A61N 1/372; A61N 1/375; A61N 1/378; A61N 1/38; A61N 1/39; A61N 1/40; G06F 17/00

(52) U.S. Cl. 607/20

(57) ABSTRACT

A medical cart, which includes respiration and cardiac functionality, can readily be transported to a patient. The cart may contain separable respiratory and cardiac units in which the cardiac unit contains one or more items useful in treating cardiac emergencies such as cardiac arrest and the respiratory unit contains one or more items useful in treating respiratory emergencies such as a stoppage of breathing. To increase access to the items that the cart carries, parts of the cart, e.g., drawers, which contain items associated with particular medical conditions, may be isolated. As a result, multiple users, who are treating different medical conditions, can access the cart at any given time. A hood may be provided to enclose items, which were placed on top of the cart, while the cart is in transit.

### CROSS-REFERENCE TO RELATED APPLICATIONS

This application claims priority to U.S. Provisional Application Nos. 60/537,509 (filed Jan. 21, 2004) and 60/621,662 (filed Oct. 26, 2004), which are incorporated herein by reference in their entirety.

### BACKGROUND

Medical carts are often used in hospitals to respond to a patient emergency (also referred to as a “crash”) such as, for example, cardiac arrest, respiratory failure, etc. These carts, which are referred to as “crash carts,” present several problems some of which are hereafter discussed.

In response to a patient crash, a nurse often grabs a crash cart and rushes in an effort to aid the patient as quickly as possible. Unfortunately, because necessary items are frequently stored on top of crash carts (to provide quick and easy access to them), the items are susceptible to falling off the cart while in rapid transit from the stored location to the patient. If a necessary item falls of the cart, two things can happen. At best, if the nurse notices the item fall off the cart, the nurse must stop and retrieve the item. And, at worst, if the nurse fails to notice the item fall off the cart, the nurse may get to the patient only to discover the necessary item was lost in transit.

Another related problem associated with conventional crash carts is that items stored on the tops of the carts tend to be removed, i.e., nurses will borrow items from the carts and forget to return or replenish them. Accordingly, when an emergency occurs, the responding nurse may be missing one or more items needed for the emergency. As a result, precious time may be lost and death may even occur as a result of the missing item(s).

In hospital wards in which patient crashes do not frequently occur, crash carts may sit idle for prolonged periods of time. As a result, dust and dirt may collect on the tops of the carts. In an effort to keep dust and dirt off the items, nurses working in these wards typically put plastic covers (or even polyliners) over the carts. Of course, when the carts are needed to respond to an infrequent patient crash, the covers must first be removed. Moreover, after removing the cover, the nurse must make sure that a necessary item was not inadvertently removed along with the cover. The time it takes to remove the cover and to check that all necessary items are present delays the response, thereby risking the patient's health (at best) or life (at worst).

Due to (a) the number of medical personnel (both doctors and nurses) that need to use a cart during a patient crash and (b) the number of medical items and/or medication that need to be carried by the cart, organization is often a problem. The access of necessary medical personnel to the cart may be impeded by other medical personnel working on the patient. Moreover, time spent looking for an item (or waiting for another user, who is also using the cart, to move away from a location in which the item is housed) impedes the user's ability to treat a patient, thereby again putting the patient's heath (or life) at risk.

Finally, as a result of the number of items and/or medications that need to be transported in crash carts, these carts are often very bulky. In hospitals that have smaller rooms (and/or when there are, for example, ten healthcare professionals trying to work around the cart in the same room), the size of the cart can be an issue.

In light of the foregoing, what is needed is a crash cart that addresses one or more of the aforementioned problems.

### SUMMARY

An aspect of the present invention relates to a medical cart comprising a cardiac unit and a respiratory unit. The cardiac unit includes rolling members that permit the cardiac unit to be rolled to a predetermined location, and a cardiac storage structure configured to contain at least one cardiac item. The respiratory unit includes rolling members that permit the respiratory unit to be rolled to the predetermined location, and a respiratory storage structure configured to contain at least one respiratory item. The medical cart also includes a mechanism configured to releasably join the cardiac unit and the respiratory unit such that the cardiac and respiratory units can be jointly rolled to the predetermined location, and configured to permit the cardiac and respiratory units to be separated at the predetermined location.

Another aspect of the present invention relates to a medical cart comprising a first unit and a second unit. The first unit is configured to transport at least one item associated with a first medical condition, wherein the first unit has a first visual guide identifying the first unit as transporting items for treating the first medical condition. The second unit is configured to transport at least one item associated with a second medical condition, the second unit being joined to the first unit, wherein the second unit has a second visual guide identifying the second unit as transporting items for treating the second medical condition.

Another aspect of the present invention relates to a medical cart comprising a mobile base and a plurality of drawers supported by the mobile base and being configured to contain at least one of cardiac and respiratory items, each of the drawers having a front side and a rear side. The medical cart also includes at least one cabinet hingedly connected to the mobile base, wherein the cabinet is configured to swing between a closed position, in which the cabinet blocks access to the front sides of the drawers, and an open position, in which the cabinet does not block access to the front side of the drawers.

Another aspect of the present invention relates to a medical cart comprising a plurality of drawers configured to contain items associated with at least one medical condition, wherein each of the drawers has a front side and a rear side. The medical cart also includes a mobile base for supporting the plurality of drawers, wherein the mobile base is configured such that the drawers can be opened by pulling the front side of the drawers and by pulling the rear side of the drawers.

Another aspect of the present invention relates to a medical cart comprising a base comprising rolling members that permit the base to be rolled to a location, a plurality of drawers configured to contain items associated with at least one medical condition, a top shelf positioned at least partially over the drawers, and a retractable hood that is movable between an open position, in which the top shelf is accessible to a user and a closed position, in which access to the top shelf is blocked.

Yet another aspect of the present invention relates to a medical cart comprising a first unit positioned at a front end of the cart, the first unit including a first storage structure configured to contain at least one item associated with a first medical condition and to permit access to the at least one item associated with the first medical condition from the front end of the cart. The medical cart also includes a second unit positioned at a rear end of the cart, the second unit including a second storage structure configured to transport at least one item associated with a second medical condition and to permit access to the at least one item associated with the second medical condition from the rear end of the cart.

These and other features, aspects, and advantages of the present invention will become more apparent from the following description, appended claims, and accompanying exemplary embodiments shown in the drawings.

### BRIEF DESCRIPTION OF THE DRAWINGS

FIG. 1A is a front perspective view of a first embodiment of a medical cart according to the present invention in which the cart comprises separable cardiac and respiratory units;

FIG. 1B is a rear perspective view of the cart shown in FIG. 1A;

FIG. 1C is a front perspective view of the cart shown in FIG. 1A with the cardiac and respiratory units separated;

FIG. 1D is an underside view of the cardiac unit of the cart shown in FIG. 1A, showing a foot pedal actuation mechanism having a toe plate actuator in a non-actuated state;

FIG. 1E is an underside view of the cardiac unit shown in FIG. 1D in which the toe plate actuator is in an actuated state;

FIG. 1F is an underside view of the respiratory unit of the cart shown in FIG. 1A, the figure showing an engagement mechanism configured to engage releasably the foot pedal actuation mechanism of the cardiac unit shown in FIGS. 1D-1E;

FIG. 1G is a close-up, rear perspective view of the units of the cart shown in FIG. 1A showing a rear engagement mechanism;

FIG. 1H is an underside view of the units shown in FIGS. 1D and 1F in a joined state;

FIG. 2A is a front perspective view of a second embodiment of a medical cart according to the present invention in which drawers are accessible from both the front and the back sides of the cart;

FIG. 2B is a rear perspective view of the cart shown in FIG. 2A;

FIG. 2C is a perspective view of the cart shown in FIG. 2A showing various of the drawers being opened toward the front side of the cart and various of the drawers being simultaneously opened toward the rear side of the cart;

FIG. 3 is a perspective view of a third embodiment of a medical cart according to the present invention in which the cart comprises distinct, but joined, cardiac and respiratory units;

FIG. 4A is a perspective view of a fourth embodiment of a medical cart according to the present invention in which a cardiac unit and a respiratory unit are provided back-to-back;

FIG. 4B is a perspective view of the cart shown in FIG. 4A showing the respiratory unit;

FIG. 4C is a perspective view of the cart shown in FIG. 4A showing the cardiac unit;

FIG. 5A is a perspective view of a fifth embodiment of a medical cart according to the present invention in which cabinets are closed, thereby covering internal, central drawers;

FIG. 5B is a perspective view of the cart shown in FIG. 5A with the cabinets opened, thereby exposing the internal, central drawers;

FIG. 6A is a perspective view of a sixth embodiment of a medical cart according to the present invention in which a retractable hood is in a closed position, thereby covering a top shelf;

FIG. 6B is a perspective view of the cart shown in FIG. 6A with the retractable hood in an open position, thereby exposing the shelf;

FIG. 6C is an exploded, perspective view of the cart shown in FIG. 6A, showing a track in which the hood is configured to move;

FIG. 6D is a cross-section of the cart shown in FIG. 6A taken along line D-D; and

FIG. 6E is a cross-section of the cart shown in FIG. 6B taken along line E-E.

### DETAILED DESCRIPTION

Presently preferred embodiments of the invention are illustrated in the drawings. An effort has been made to use the same or like reference numbers throughout the drawings to refer to the same or like parts.

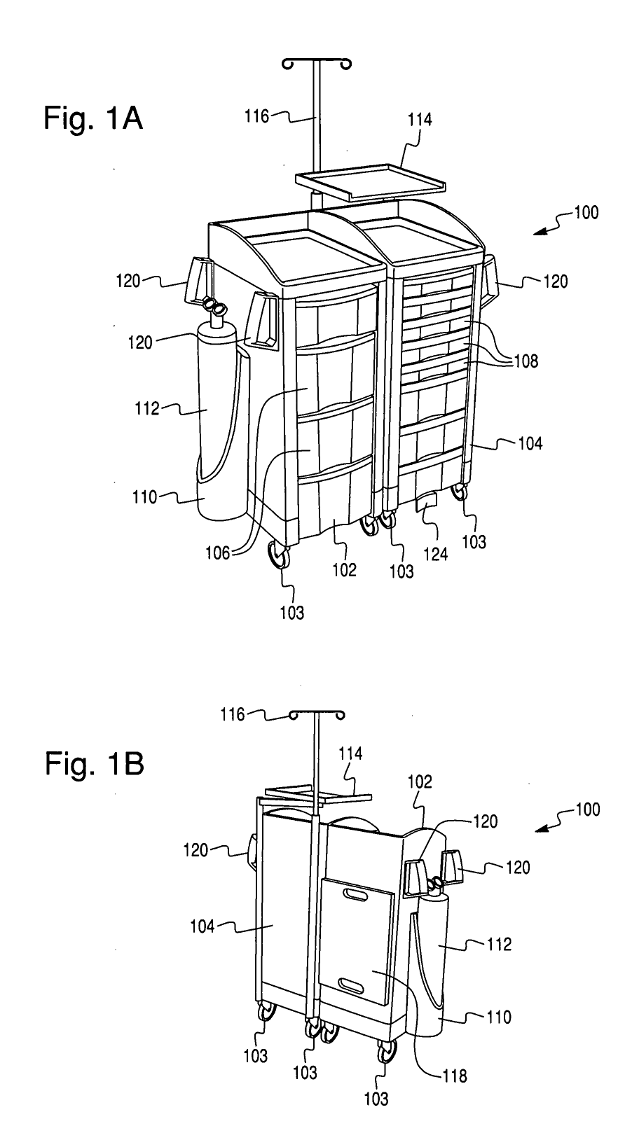

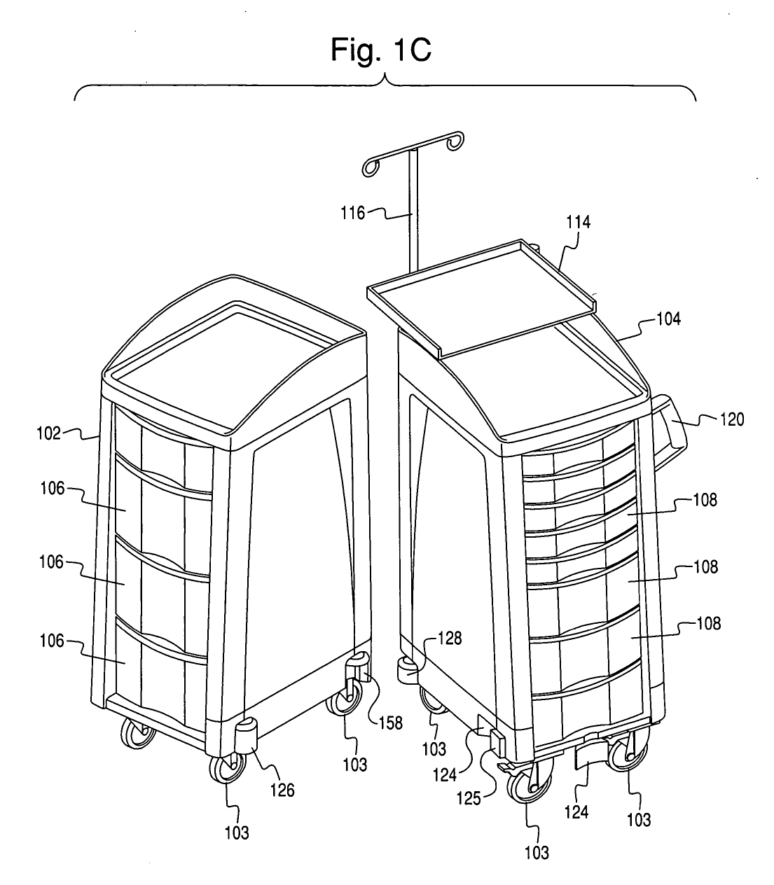

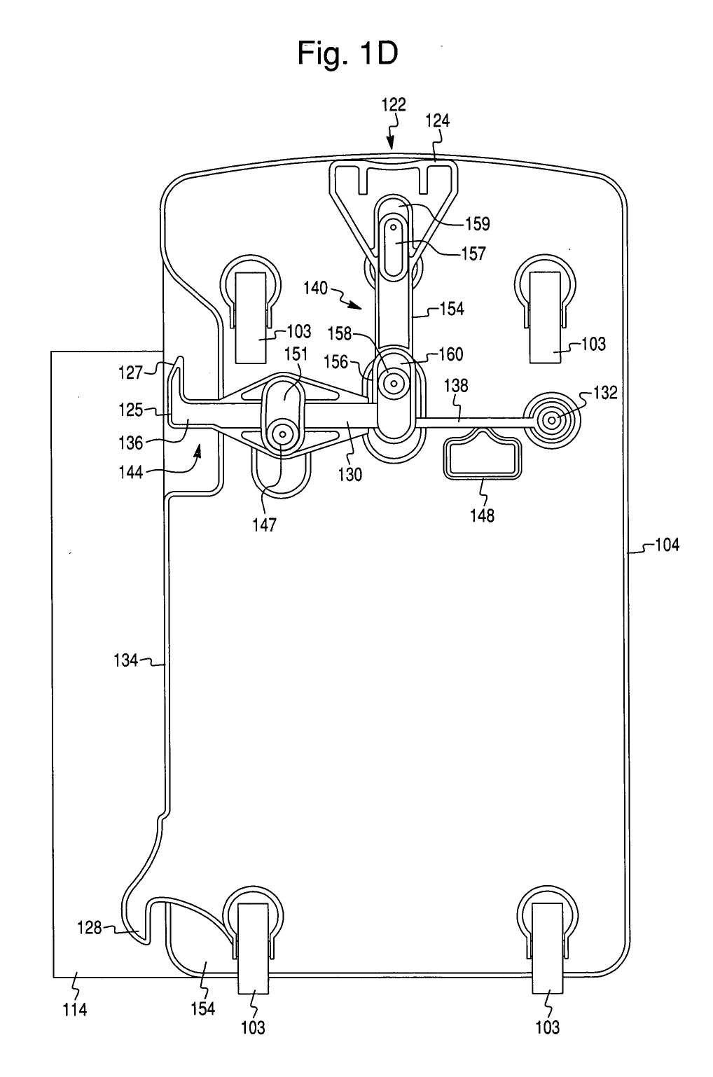

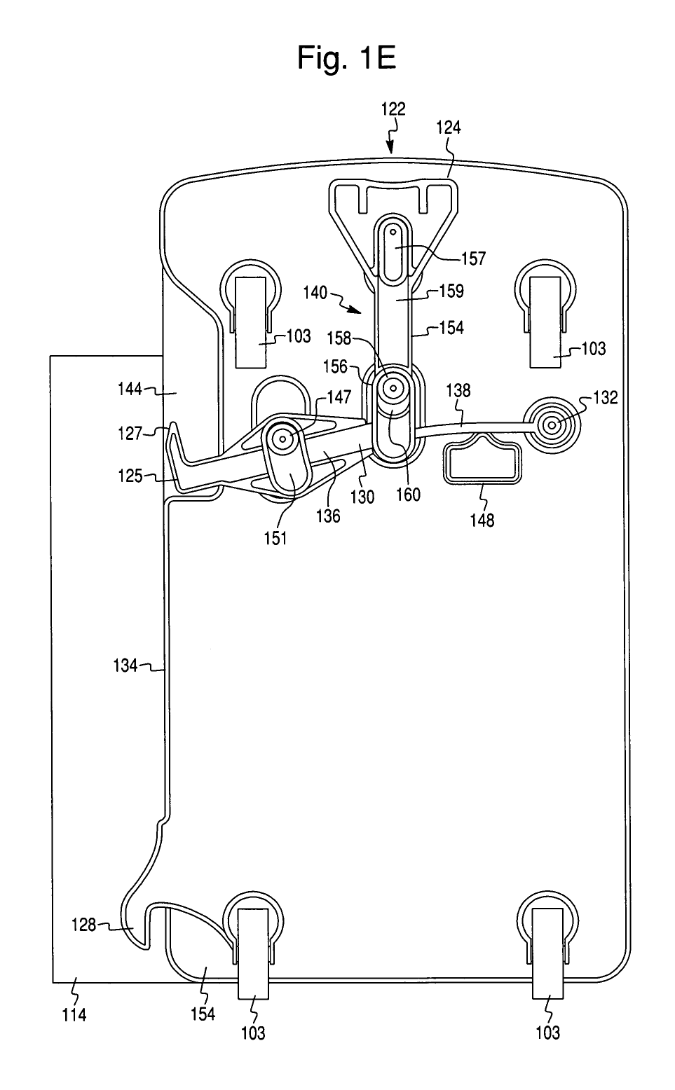

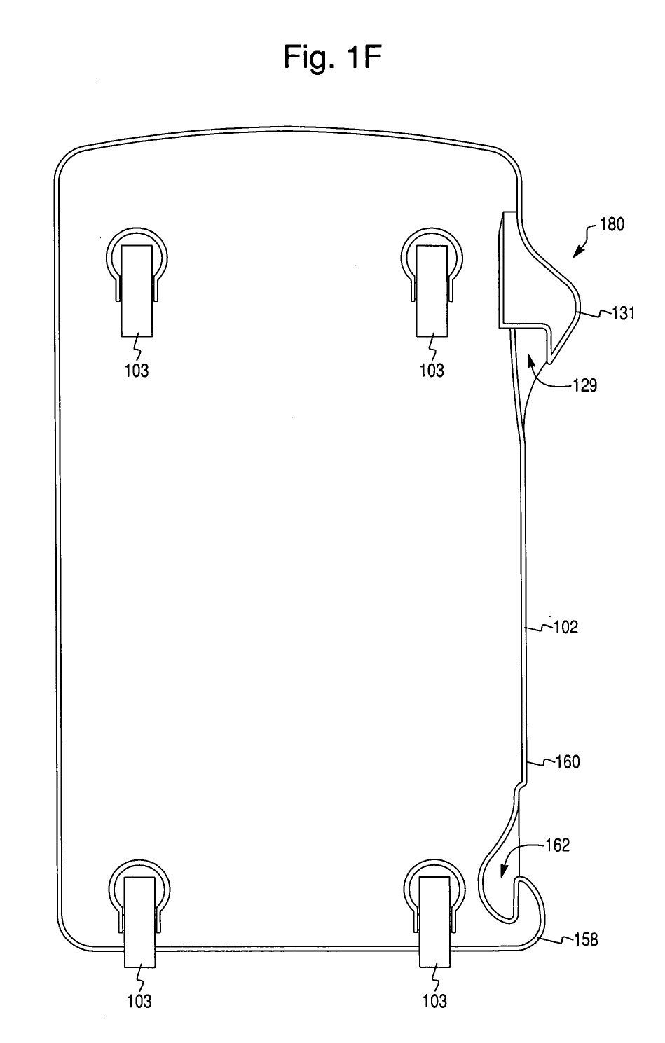

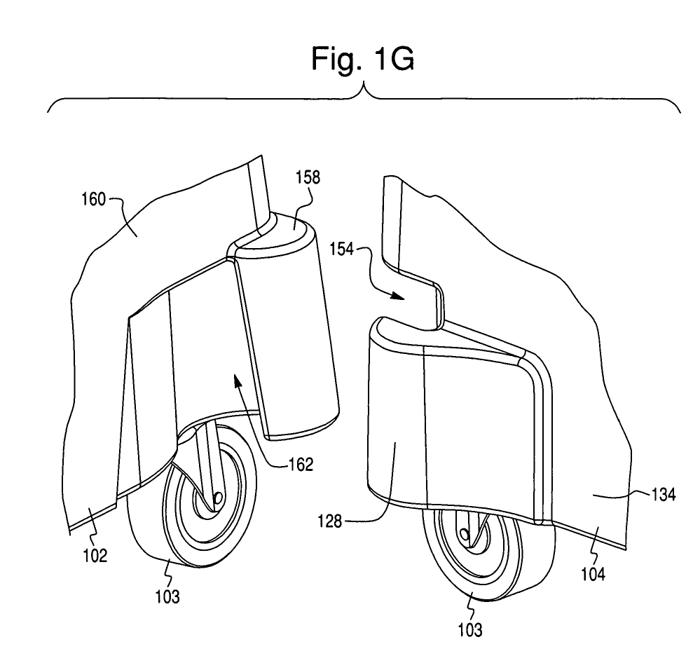

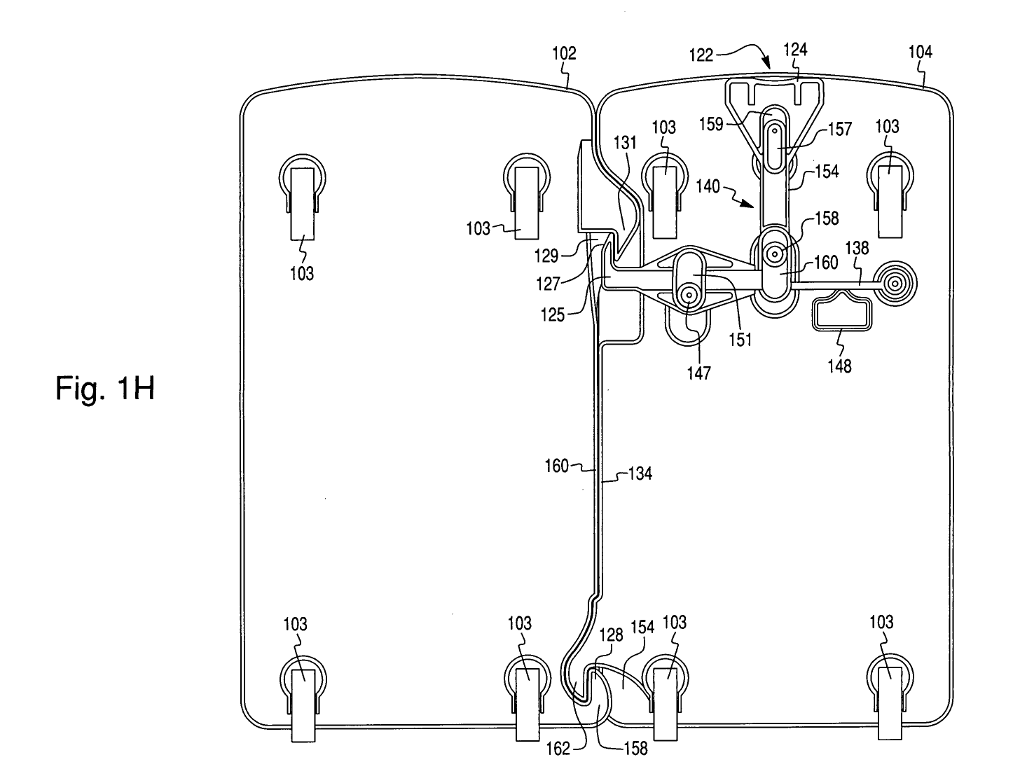

A first embodiment of a crash cart is shown in FIGS. 1A-1H. The crash cart 100 includes separable respiratory and cardiac units 102, 104, each of which is supported by rolling members 103. As a result, when the units 102, 104 are joined, the entire cart 100 can be rushed, via the rolling members (e.g., wheels such as casters) 103, to a patient, who is in need of emergency assistance. When the cart 100 arrives at the patient, the respiratory and cardiac units 102, 104 may be separated. The separation of the units 102, 104, which is later described in detail, enables increased access to the cart 100, which is extremely beneficial in some environments in which, for example, space is limited.

As a result of the separability of the units 102, 104, the respiratory unit 102 may, for example, be positioned on one side of the patient's bed and the cardiac unit 104 may be positioned on the other side of the patient's bed. Consequently, respiratory personnel can be separated from cardiac personnel. By way of further example, if there were four respiratory individuals and four cardiac individuals, all eight individuals would be vying for access to a conventional crash cart in which respiratory and cardiac functionality is combined. In contrast, if the units 102, 104 of this cart 100 are separated, the four respiratory individuals only need access to their unit 102 and the four cardiac individuals only need access to their unit 104. As a result, whereas eight individuals would vie for access to a conventional cart, in this embodiment two separate sets of four individuals would vie for access to a particular unit 102, 104.

The respiratory and cardiac units 102, 104 respectively include storage structures that are configured to carry items associated with particular medical conditions. For example, drawers 106 in the respiratory unit 102 can be configured to contain medical items associated with respiratory conditions, e.g., a stoppage of breathing. Such items might include oxygen masks, airway tubes, laryngoscopes, laryngoscope blades, resuscitators, intubation kits, suction machines, etc. Similarly, drawers 108 in the cardiac unit 104 can be configured to contain medical items associated with cardiac conditions, e.g., a cardiac arrest. Such items might include vials of adrenaline, defibrillators, medications, syringes, intravenous (“I.V.”) needles, I.V. starter kits, etc.

In addition to the drawers 106, 108, each of the units can also be configured to carry other items associated with particular medical conditions. For example, the respiratory unit 102 may include a container 110 that is sized to hold an oxygen bottle 112, as shown.

Moreover, as shown in FIG. 1B, the respiratory unit 102 may also have a rigid backboard 118, which is configured to be placed on a patient's back, thereby providing a hard flat surface for chest compressions (because soft patient beds often do not provide adequate support for this procedure). The backboard 118 may be, for example, hanged on the respiratory unit 102, as shown. Similarly, the cardiac unit 104 may contain a defibrillation platform 114 on which a defibrillator (not shown) can be positioned. Moreover, either (or both) of the units 102, 104 may contain an intravenous administration member 116 (which can be in the form of a pole and is sometimes referred to as a “tree”) and/or handles 120 that a user can use to direct the cart 100 or a separated unit 102, 104.

To enhance the functionality of this cart 100, the respiratory and cardiac units 102, 104 may include respective first and second visual guides, which identify the medical condition to be treated by the unit. The visual guide can be a color. For example, the faces of drawers 106 and/or sidewalls of the respiratory unit 102 may be blue in color. Similarly, the faces of the drawers 108 and/or sidewalls of the cardiac unit 104 may be red in color. As a result, if a doctor/nurse rushes into a room in which a patient is being treated for a medical emergency, the doctor/nurse can readily determine which unit 102, 104 will contain the medical items necessary to treat the medical conditions to which the doctor/nurse is responding.

As previously mentioned, the units 102, 104 may be joined (FIG. 1A) and separated (FIG. 1C). The mechanism by which the units 102, 104 may be releasably joined is shown in FIGS. 1D-1H. A description of a similar mechanism can be found in U.S. Pat. No. 5,740,625, which was issued to Presnell et al., and is incorporated herein by reference in its entirety.

Either of the respiratory and cardiac units 102, 104 can have a foot actuated locking mechanism 140 there under and the other unit 102, 104 can have a catch system 180 that is configured to releasably engage the locking mechanism 140. As shown, the locking mechanism 140 is positioned under the cardiac unit 104 and the catch system 180 is positioned under the respiratory unit 102. The locking mechanism 140 will be described with respect to FIGS. 1D-1E and catch system 180 will be described with respect to FIG. 1F.

With respect to FIGS. 1D-1E, the locking mechanism 140 includes an arm member 130 that extends from a hub 132 to an outer edge 134 of the cardiac unit 104. The arm member 130 may be constructed of an elastic material (e.g., nylon, acetyl, and delrin, etc.) such that after the arm member 130 bends, it can regain its original, generally linear shape.

The arm member 130 has a front portion 136 and a rear portion 138. One of the primary functions of the rear portion 138 is to act as a pivot for the arm member 130. One of the primary functions of the front portion 136 is to assist in securing the cardiac unit 104 to the respiratory unit 102, as later discussed with respect to FIG. 1H.

Although the arm member 130 may be formed of two or more separate members secured together, in the shown embodiment, the arm member 130 is a one piece member having the front and rear portions 136, 138 integrally molded. The front portion 136 is slideably secured to the underside of the unit 104 via a projection 147. The projection 147 is fixedly secured to the underside of the cardiac unit 104 and is retained within a slot 151 in the front portion 136 of the arm member 130. As a result of the slot 151, the arm member front portion 136 may slide along the projection 147 such that the arm member 130 moves between a locked position (FIG. 1D) and an unlocked position (FIG. 1E), as later discussed in detail. In contrast to the slideable nature of the front portion 136, the rear portion 138 of the arm member 130 is fixedly secured (e.g., by nuts, bolts, screws, etc.) to the underside of the cardiac unit 104.

The front and rear portions 136, 138 of the arm member 130 have different dimensions, in part due to the different functions they perform. Specifically, the rear portion 138 generally has smaller dimensions for width and height as compared to the front portion 136, thereby providing the arm member 130 with proper flexibility. The length of the arm member 130 may range, for example, between approximately 8 and 11 inches, and the width ranges between approximately 0.25 and 2 inches, and the height ranges between approximately 0.25 and 1 inches.

A second projection 148 on the underside of the cardiac unit 104 is oriented such that the approximate center of the rear portion 138 of the arm member 130 contacts the approximate center of the projection 148. As a result, the projection 148 assists in controlling the bending of arm member 130, when a user depresses a foot pedal 122 that forms part of a foot actuated locking mechanism 140.

As shown, the foot pedal 122 generally includes of a toe plate 124, an intermediate portion 154, and a rear portion 156, which may be integrally molded or secured together by a variety of securing means (e.g., nuts, bolts, screws, etc.). A user may control the movement of the arm member 130 (and ultimately the timing of the detachment of the cardiac unit 104 from the respiratory unit 102), by selectively controlling when pressure is applied to the toe plate 124 of the foot pedal 122.

Similar to the front portion 136 of the arm member 130, the toe plate 124 is slideably secured to the underside of the cardiac unit by means of a stationary projection 157. The stationary projection is configured to slide within a slot 159 formed in the toe plate 124, when force is applied to (or released from) the toe plate 124.

The foot pedal intermediate portion 154 is secured to a rear side of the toe plate 124 and to a front side of the rear portion 156 of the foot pedal 122. The foot pedal rear portion 156 is oriented approximately adjacent to the arm member 130 and will directly contact the arm member 130 in response to force applied to the foot plate 124, as illustrated in FIG. 1E.

Similar to the front portion 136 of the arm member 130 and the toe plate 124, the foot pedal rear portion 156 may also be slideably secured to the underside of cardiac unit 104 by a variety of means one of which, as shown, includes a projection 158. The projection 158 is slideably retained within a slot 160 disposed within the rear portion 156 of the foot pedal 122, thereby limiting the movement of the foot pedal 122 to the size of the slot 160. As a result, the foot pedal 122 can slideably move between at least a locked position (FIG. 1D) and an unlocked position (FIG. 1E).

In the locked position, the arm member 130 is generally in a relaxed state such that both the front portion 136 and the rear portion 138 of the arm member are substantially linearly aligned. In contrast, when the toe plate 124 of the foot pedal 122 is depressed, the toe plate 124 causes the intermediate portion 154 and, in turn, the rear portion 156 to slide rearward, i.e., the stationary projections 157, 158 respectively slide in their corresponding slots 159, 160. As the rear portion 156 of the foot pedal 122 moves, the rear portion 138 of the arm member 130 is forced to bend elastically at the point at which it contacts stationary projection 148. As a result, the following occurs: (a) the arm member 130 is forced to bend such that the front portion 136 thereof and the rear portion 138 no longer linearly aligned (as shown in FIG. 1E); (b) as the arm member 130 bends, the stationary projection 147 slides within the slot 151 in the front portion 136 of the arm member 130; and (c) the front portion 136 of the arm member 130 moves rearwardly in a recess 144 formed in the underside of the cardiac unit 104.

The front portion 136 of the arm member 130 is generally L shaped and defines a hook 125, which resides in the recess 144 in the cardiac unit 104. The L-shaped hook 125 is configured to engage an L-shaped shaped recess 129 (FIG. 1F), which is formed in a projection 131 on a lower side of the respiratory unit 102 and which is sized to receive the hook 125 of the arm member front portion 136 (FIG. 1H), as will be later discussed.

With respect to FIGS. 1E and 1F, a rear engagement mechanism generally consists of two cooperating elements: (a) a recess 154, disposed adjacent the outer edge 134 of the cardiac unit 104; and (b) a hook-like rear projection 158 extending in an outwardly direction from an outer edge 160 of the respiratory unit 102. The recess 154 on the cardiac unit 104 is enclosed by a hook-like projection 128 and the hook-like rear projection 158 on the respiratory unit 102 encloses a rear recess 162. Further, as shown best in FIG. 1G, the recess 154 formed in the cardiac unit 104 is sized to receive the hook-like projection 158 on the respiratory unit 102 and, similarly, rear recess 162 on the respiratory unit 102 is sized to receive the hook-like projection 128 on the cardiac unit 104.

To attach the respiratory unit 102 to the cardiac unit 104, first the user rolls the respiratory unit 102 (or the cardiac unit 104) so that the projection 128 of the cardiac unit 104 is received in the rear recess 162 in the respiratory unit 102 (at the same time the projection 158 on the respiratory unit 102 will be received in the recess 154 in the cardiac unit 104). Subsequently, the front portions of the respiratory and cardiac units 102, 104 are rolled toward each other. When the curved outer side of the projection 131 extending from the outer edge 160 of the respiratory unit contacts a sloped side 127 of the L-shaped hook 125 of the front portion 136 of the arm member 130, the arm member front portion 136 will be rearwardly driven to the position shown in FIG. 1E. A further movement of the units 102, 104 toward each other will cause the L-shaped hook 125 of the front portion 136 of the arm member 130 to snap (under the elastic force acting on the rear portion 138 of the arm member 130) into the corresponding recess 129 in the respiratory unit 102. As a result, the units 102, 104 will be releasably joined in the manner shown in FIG. 1H.

The user may easily separate the respiratory and cardiac units 102, 104. To separate the units 102, 104, the user must exert a slight rearward pressure on the outer edges of the units 102, 104 while simultaneously actuating the foot pedal 122 by rearwardly depressing the toe plate 124. As the toe plate 124 is depressed, the foot pedal 122 slideably moves in a rearwardly direction. The sliding movement of the foot pedal 122 is limited by the size of the slots 159, 160. When the foot pedal 122 is at its most rearward portion, the foot pedal rear portion 156 exerts pressure against the front portion 136 of the arm member 130. In addition, the sliding movement of the foot pedal 122 also causes the rear portion 138 of the arm member 130 to conform its shape to that of the projection 148. Both of these events cause the front portion 136 of the arm member 130 to bend in a generally rearwardly direction (into the position shown in FIG. 1E); the rearward bending of the arm member front portion 136 is limited by the size of the slot 151. As the front portion 136 of the arm member 130 bends in a generally rearwardly direction, the hook 125 subsequently becomes disengaged from the projection 131 in the corresponding recess 129 in the respiratory unit 102. Thus, the front portions of the units 102, 104 may be easily disengaged by rolling the front portions of the units 102, 104 away from each other. Finally, to completely disengage the units 102, 104, the rear portions of the units 102, 104 must be disengaged by withdrawing the projection 128 on the cardiac unit 104 from the recess 162 in the respiratory unit 102 (and at the same time the withdrawing the projection 158 on the respiratory unit 102 from the recess 154 in the cardiac unit 104).

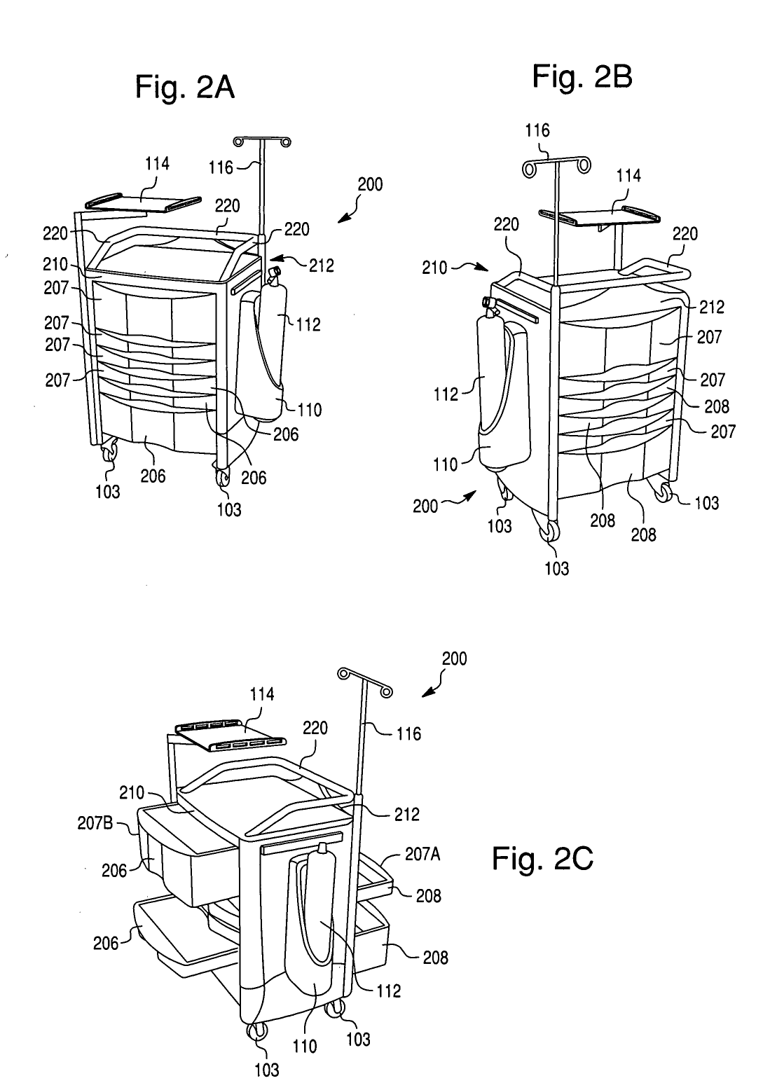

A second medical cart 200 embodiment according to the present invention is described with respect to FIGS. 2A-2C. In this embodiment, the medical cart 200 does not have separable respiratory and cardiac units. However, this medical cart 200, like the previously described medical cart 100, provides increased access for medical personnel.

Access is increased by providing bidirectional drawers 207 that may be accessed from two sides of the cart, e.g., the front side 210 and back side 212 of the cart. For example, with reference to FIGS. 2A and 2B, the drawers 207 have fronts (FIG. 2A) 206 that are accessible from the front side 210 of the cart 200 and backs (FIG. 2B) that are accessible from the back side 212 of the cart 200.

As a result of the dual accessibility of the drawers 207, some medical personnel (e.g., respiratory individuals) can obtain access to some the drawers 207 from the front 210 of the cart 200 while other medical personnel (e.g., cardiac individuals) can obtain access to the remaining drawers 207 from the back 212 of the cart 200. Moreover, in contrast to conventional carts in which if a drawer is opened, access to lower drawers is blocked, in this embodiment if one drawer 207 is opened toward the front 210 of the cart 200, the immediately lower drawer 207 can be accessed from the rear 212 of the cart 200. In addition, to facilitate the process by which a particular drawer 207 is selected, some of the drawers 207 (or the faces of the drawers) may be color-coded for a first medical condition (e.g., a respiratory condition) whereas the other drawers 207 (or the faces of those drawers) may be color-coded for a second medical condition (e.g., a cardiac condition). By way of specific example and with respect to FIG. 2C, some drawers 207A may be, e.g., blue (which may be associated with a respiratory condition) whereas the other drawers 207B may be, e.g., red (which may be associated with a cardiac condition).

In addition to the foregoing, this medical cart 200, like the previously described medical cart 100, may have increased functionality. Specifically, the medical cart 200 may include rolling members 103, a defibrillation platform 114, an I.V. medication tree 116, an oxygen container 110 for holding an oxygen bottle 112, a backboard 118, and/or one or more handles 220 to facilitate directing the cart 200.

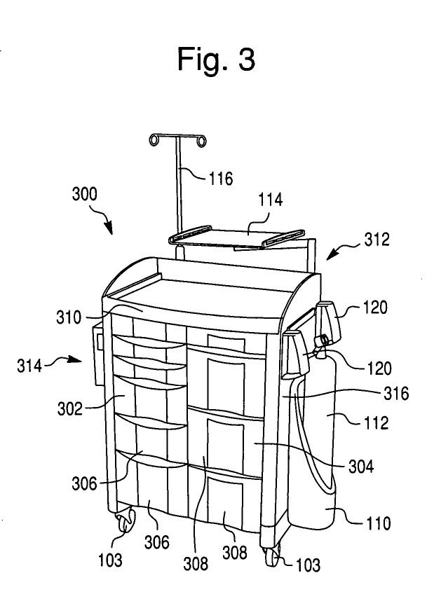

A third medical cart 300 embodiment according to the present invention, which combines some of the characteristics of each of the medical carts 100, 200 previously discussed, is shown in FIG. 3. Similar to the first medical cart 100, this medical cart 300, includes distinct respiratory and cardiac units 302, 304, which respectively have drawers 306, 308. Moreover, the respiratory and cardiac units 302, 304 may be color-coded in the manner previously described, i.e., the respiratory unit 302 (or its drawer faces) may be, e.g., blue and the cardiac unit 304 (or its drawer faces) may be, e.g., red. Unlike the first cart 100, however, the respiratory and cardiac units 302, 304 of this cart 300 are not separable from each other. However, the individual units 302, 304 reduce the time necessary to find a particular medical item because a user (e.g., a cardiac technician) will know in which set of drawers a particular item will be found. Moreover, if any of the drawers 306 in the respiratory unit 302 are opened access is not blocked to any of the drawers 308 of the cardiac unit 304.

In the shown embodiment, the individual units 302, 304 can be accessed by medical personnel standing adjacent the front side 310 of the cart 300 and/or slightly toward the sides 314, 316 of the cart 300. In some embodiments, however, the access to the drawers 306, 308 can be enhanced by enabling the drawers 306, 308 to be bidirectional similar those shown in the second medical cart 200 embodiment, i.e., the units 302, 304 may be accessible from both the front side 310 and the back side 312 of the cart 300.

In addition to the foregoing, this medical cart 300, like the previously described medical carts 100, 200, may have increased functionality. Specifically, the medical cart 300 may include rolling members 103, a defibrillation platform 114, an I.V. medication tree 116, an oxygen container 110 for holding an oxygen bottle 112, a backboard 118, and/or one or more handles 120 to facilitate directing the cart 300.

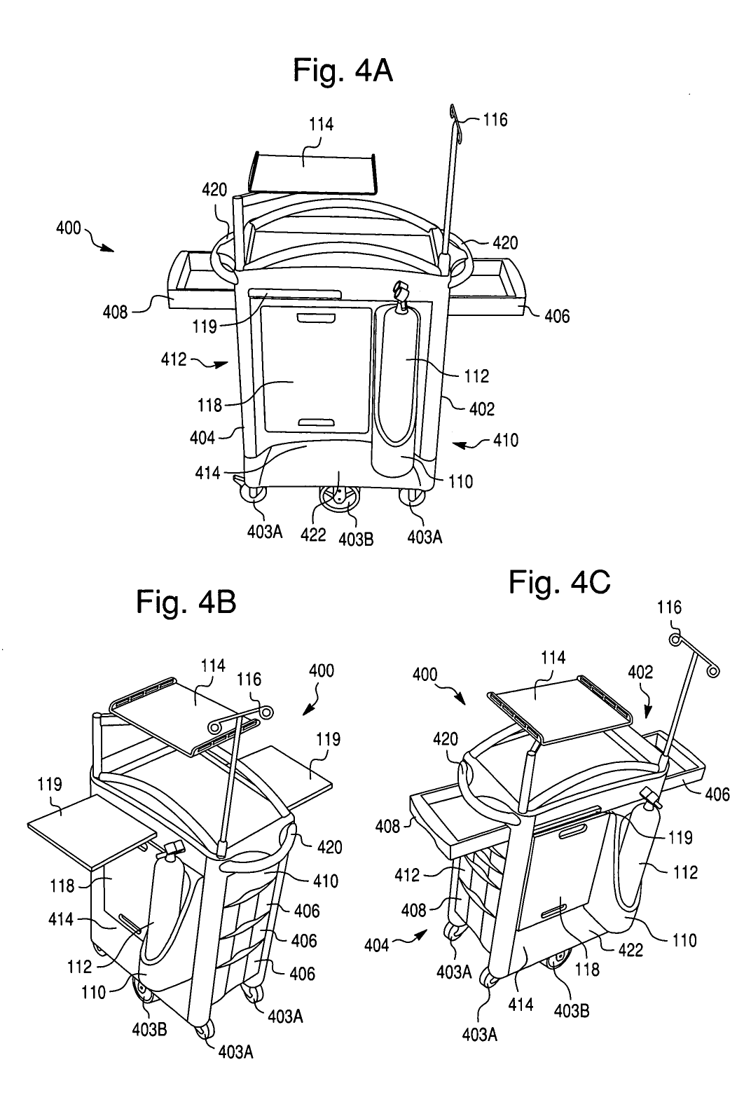

A fourth medical cart 400 embodiment according to the present invention, which is similar in some characteristics to the third medical cart 300, is shown in FIGS. 4A-4C. The medical cart 400 includes both respiratory and cardiac units 402, 404. Although the respiratory and cardiac units 402, 404 are not separable from each other, they are functionally separated from each other by being positioned back-to-back. As a result, the drawers 406 of the respiratory unit 402 are accessible from the front side 410 of the cart 400 (FIG. 4B) whereas the drawers 408 of the cardiac unit 404 are accessible from the back side 412 of the cart 400 (FIG. 4C).

Accordingly, medical personnel associated with a first medical condition (e.g., a respiratory condition) can be positioned at the front side 410 of the cart 400 and medical personnel associated with a second medical condition (e.g., a cardiac condition) can be positioned at the back side 412 of the cart 400. Moreover, to facilitate the separation of the medical personnel, the respiratory and cardiac units 402, 404 may be color-coded in the manner previously described, i.e., the respiratory unit 402 (or its respective drawer faces) may be, e.g., blue and the cardiac unit 404 (or its respective drawer faces) may be, e.g., red.

As shown best in FIG. 4A, this medical cart has a plurality of rolling members 403A, 403B. The rolling members 403A at the corners of the cart 400 may be similar to those provided on the previously described carts 100, 200, 300. More specifically, the rolling members 403A at the corners may be casters. In a center portion 422 of the sides 414, 416 of the cart 400, however, of the cart 400 may include additional rolling members 403B.

The rolling members 403B provided in the center side portions 422 may, for example, have fixed axles or may be casters. Moreover, the rolling members 403B in the center side portions 422 may have larger diameters than the rolling members 403A provided at the corners of the cart 400. As a result, if the central rolling members 403B have fixed axles, the cart 400 can be designed to teeter on those axles such that the cart 400 rests on the two center rolling members 403B and two of the corner rolling members 403A. By enabling the cart 400 to teeter in this fashion, the maneuverability of the cart 400 can be greatly enhanced. For example, in carts having four rolling members all of which are casters, when the cart rounds a corner, inertia tends to drive the cart along a tangential path, i.e., the cart may not corner well. In contrast, by replacing two of the casters with fixed axle rolling members, corners can be rounded more easily because the inertial forces are countered by the fixed axles.

In addition to the foregoing, this medical cart 400, like the previously described medical carts 100, 200, 300, may have increased functionality. Specifically, the medical cart 400 may include a defibrillation platform 114, an I.V. medication tree 116, an oxygen container 110 for holding an oxygen bottle 112, a backboard 118, and/or one or more handles 420 to facilitate directing the cart 200. In addition, the cart 400 may also include one or more retractable work surfaces 119 that are configured to slide into the sides 414, 416 of the cart 400 when not in use.

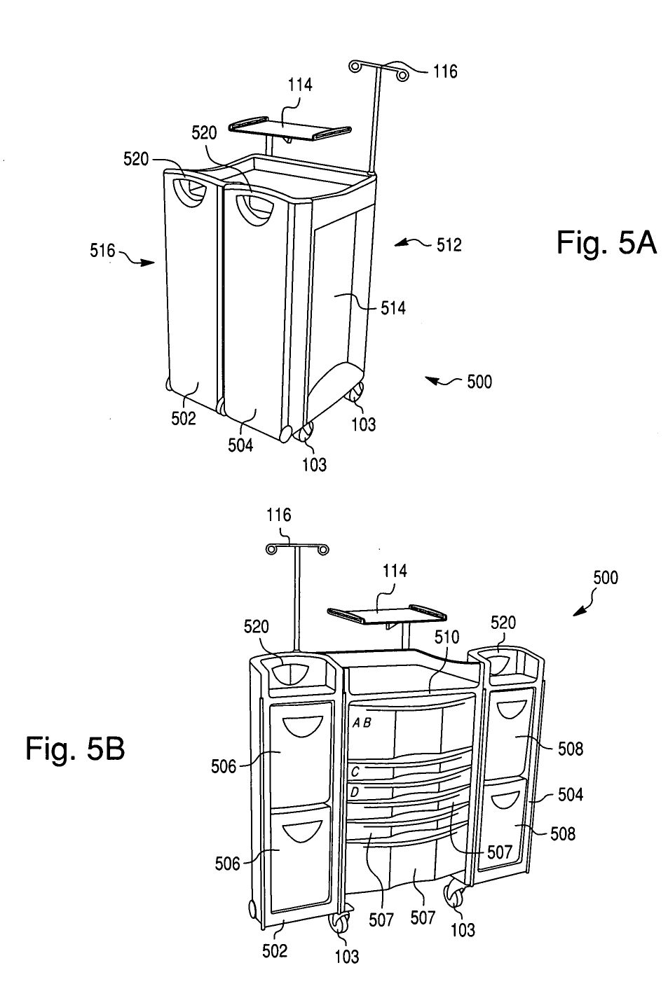

A fifth embodiment of a medical cart 500 according to the present invention is shown in FIGS. 5A-5B. In this embodiment, the medical cart 500 is, like the medical cart 100 described in the first embodiment, configured to be transported, in one configuration, to a patient crash site and subsequently changed into a second configuration. Specifically, in transit (FIG. 5A), the cart 500 can have two cabinets 502, 504 (e.g., respiratory and cardiac units) in a closed position. The cabinets contain storage members, such as drawers.

When the cart 500 arrives at a predetermined location, e.g., the site of a patient crash, the cabinets 502, 504 can be opened, as shown in FIG. 5B. When the cabinets 502, 504 are opened, the overall width of the cart 500 substantially doubles. This increased width facilitates access to the various drawers 506, 508 in the cabinets, respectively, as well as central drawers 507 in the front side 510 of the cart 500.

As a result of the increased width, medical personnel associated with a first medical condition (e.g., a respiratory condition) can work with the drawers 506 of one of the cabinets 502 while medical personnel associated with a second medical condition (e.g., a cardiac condition) work with the drawers 508 of the other cabinet 504. Further, the central drawers 507 in the front side 510 of the cart 500 may be shared by the two sets of medical personnel.

To facilitate directing the sets of medical personnel, one of the cabinets 502 (including drawers 506) and/or some of the central drawers 507 in the front side 510 of the cart 500 may be color-coded (e.g., blue) to indicate an associated first medical condition. Similarly, the other cabinet 504 (including drawers 508) and/or some of the central drawers 507 in the front side 510 of the cart 500 may be color-coded (e.g., red) to indicate an associated second medical condition. Finally, like the carts 200, 300 shown in FIGS. 2A-3, the central drawers 507 visible on front side 510 of the cart 500 may, in some embodiments, also be accessible via the back side 512 of the cart, i.e., the central drawers 507 may be bidirectional.

As shown in FIG. 5B, three of the central drawers 507 are respectively labeled “AB,” “C,” and “D” (hereafter referred to as “ABCD drawers 507”). The ABCD drawers 507 can be sized to hold medical items associated with a medical protocol acronym (“A-B-C-D”), which is formed of the first letters of the following four concepts: (1) “Airway”; (2) “Breathing”; (3) “Circulation”; and (4) “Defibrillation.” During a patient crash, the A-B-C-D acronym is applied in sequence as follows. First, medical personnel check to see if the patient's airway is blocked. Second, if the airway is open, they check to see if the patient is breathing. Third, if the patient is breathing, they check the patient's blood circulation. And, fourth, if the patient's blood is not circulating (which may be indicative of cardiac arrest), defibrillation may be applied.

In light of these steps, the ABCD drawer 507 labeled “AB” can contain all of the medical items (e.g., airway tubes, laryngoscopes, laryngoscope blades, resuscitators, pumps, airway masks, intubation kits) useful both to check a patient's airway and to treat a patient who has stopped breathing as a result of a clogged airway. If the patient is breathing, the medical personnel can turn to the ABCD drawer 507 labeled “C” for medical items (e.g., blood pressure cuffs and associated inflators, stethoscopes, medications, IV tubes, IV starter kits, needles, syringes, etc.) useful to check if a patient's blood is circulating (or whether the patient is, for example, in a state of cardiac arrest). Then, if its determined that the patient's blood is not circulating, the medical personnel can turn to the ABCD drawer 507 labeled “D” for items such as, e.g., a defibrillator, defibrillator pads, electrodes, batteries, etc.

Conventional carts usually place the items necessary for the AB sequence of the ABCD protocol toward the bottom of crash carts because they tend to be relatively large in size. The “AB” drawer typically has a larger vertical dimension than the “C” and “D” drawers. In contrast, this crash cart 500 is ergonomically designed by placing the AB sequence of the ABCD drawers 507 at the top of the cart 500. As a result, the amount of bending and reaching that medical personnel must undergo to retrieve the necessary, most frequently used items is reduced, because a majority of crashes are a result of respiratory failure only.

In addition to the foregoing, this medical cart 500, like the previously described medical carts 100, 200, 300, 400, may have increased functionality. Specifically, the medical cart 500 may include a defibrillation platform 114 on which a defibrillator (which may be removed from the ABCD drawer 507 labeled “D”) may be positioned. Similarly, this medical cart 500 also may contain an oxygen container 110 for holding an oxygen bottle 112. The oxygen bottle 112 could be used in conjunction with an oxygen mask (not shown) taken from the ABCD drawer 507 labeled “B.” In addition, this medical cart 500 may also contain: (a) one or more handles 520 (which may be formed in the cabinets 502, 504, as shown) to facilitate directing the cart 500; (b) an I.V. medication tree 116; (c) rolling members 103; and/or (d) a backboard 118 hanged on the back side 512 of the cart 500 (if the drawers 507 are unidirectional) or to either of the sides 514, 516 of the cart 500 (if the drawers 507 are bidirectional).

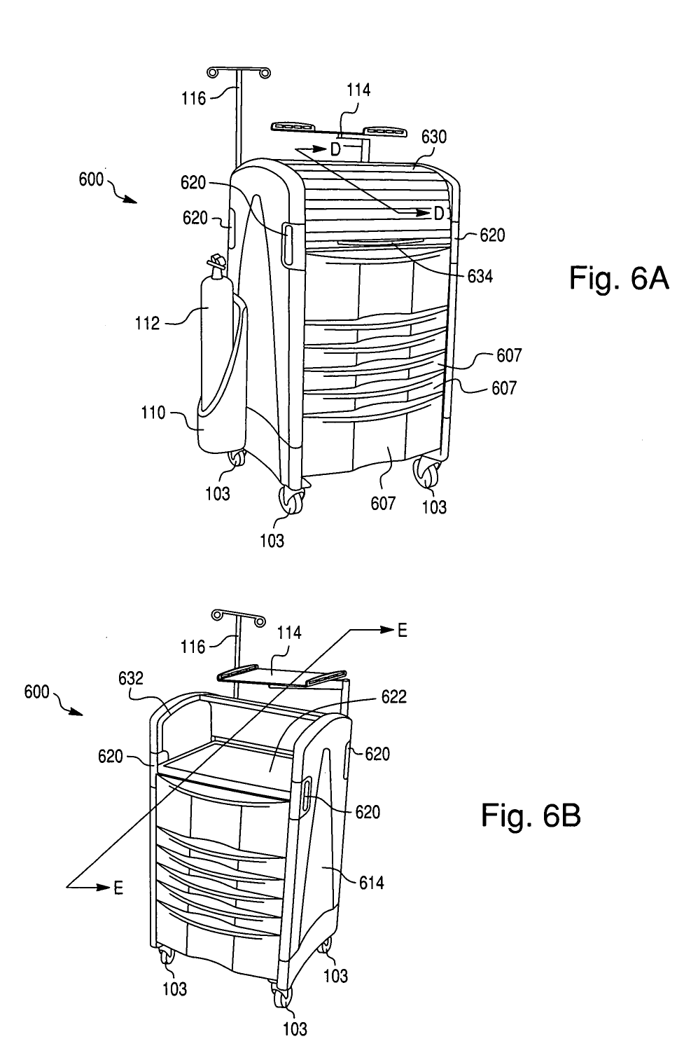

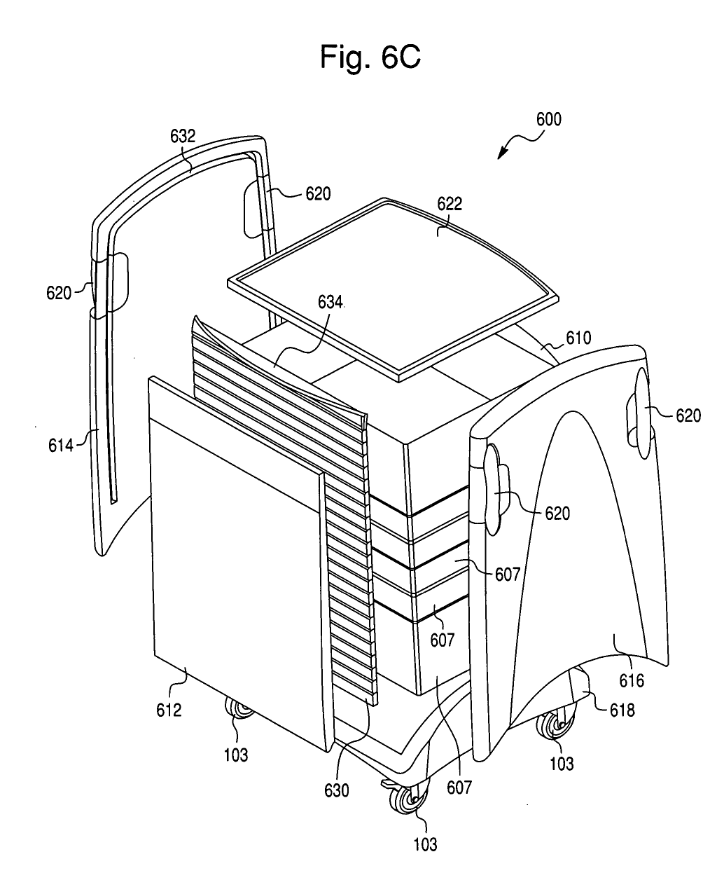

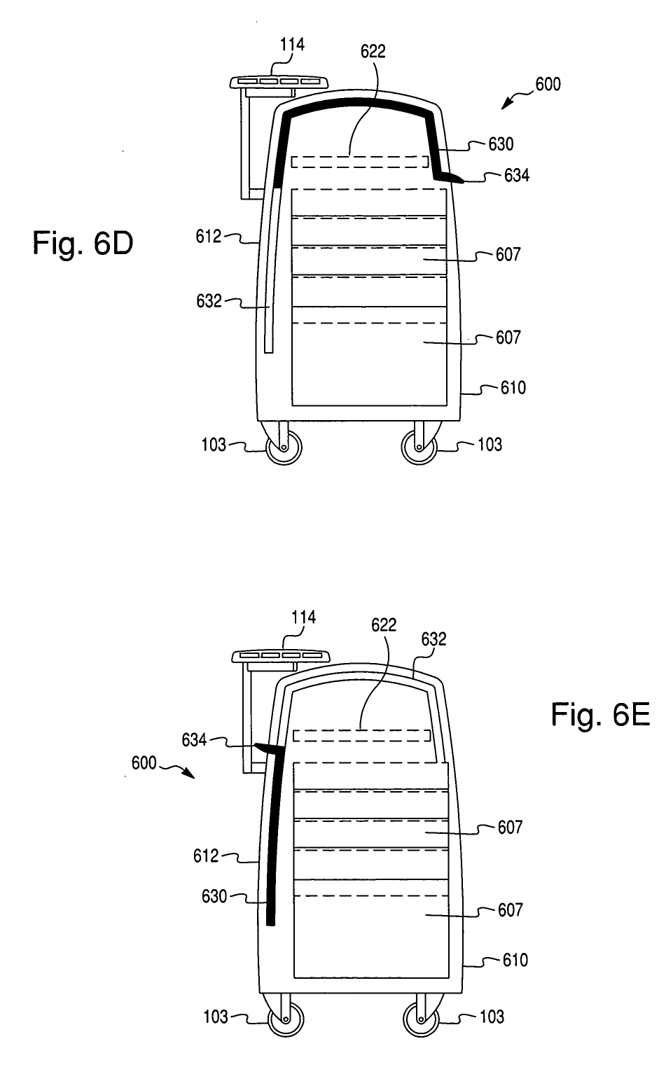

A sixth embodiment cart 600 according to the present invention is shown in FIGS. 6A-6E. The cart 600 includes a flexible, retractable hood 630 that is configured to be moved between a closed position (FIG. 6A) and an open position (FIG. 6B), as will later be described in detail. Preliminarily, however, it should be noted that this cart 600, like previous embodiments, may include an oxygen holder 110 for carrying an oxygen bottle 112, rolling members 103, an I.V. tree 116, one or more handles 620, and one or more drawers 607, which may be color-coded to correspond to particular medical conditions (e.g., respiratory and cardiac conditions).

The retractable hood 630 will be discussed primarily with respect to FIG. 6C-6E. As shown in exploded view in FIG. 6C, the cart 600 is formed of: (a) a base 618 (to which the rolling members 103 are affixed); (b) sidewalls 614, 616; (c) a back wall 612; (d) a top shelf 622; and (e) a plurality of drawers 607, the fronts of which define a front side 610 of the cart 600.

The sidewalls 614, 616 contain conventional drawer guides (not shown) on which the drawers 607 are configured to slide. Also formed on the sidewalls 614, 616 are hood tracks 632 in which the hood 630 is configured to retract from the closed position (FIG. 6A) to the open position (FIG. 6B). The hood 630 can be formed in the manner of a conventional roll top hood.

As shown in FIGS. 6D and 6E, the hood 630 contains a handle 634. The handle 634 enables a user (e.g., medical personnel) to lift and retract the hood 630, by sliding the hood 630 in the tracks 632 in the sidewalls 614, 616, from the closed position (FIG. 6D) to the open position (FIG. 6E). Similarly, the handle 634 enables the user to pull the hood 630 from the open position to the closed position.

During a patient crash, items placed on the top shelf 622 may be secured by the hood 630 (in the closed position) so that the items are not subject to falling off of the cart 600 while the cart is in rapid transit. In contrast, when the cart 600 arrives at the site of the patient crash (and the cart 600 is stationary), the hood 630 may be retracted to the open position so that a user can access the items stored on the top shelf 622.

While the cart 600 is not in use, the hood 630 (in the closed position) serves to keep dust and debris off items stored on the top shelf 622 of the cart 600. Moreover, the hood 630 keeps the cart 600 looking neat because it conceals any loose items and clutter that may be on the cart. In addition, the closed hood 630 may serve as a deterrent to medical personnel who may want to “borrow” items on the top shelf 622 and then later forget to return or replenish them.

Although the aforementioned describes embodiments of the invention, the invention is not so restricted. It will be apparent to those skilled in the art that various modifications and variations can be made to the disclosed embodiments of the present invention without departing from the scope or spirit of the invention.

For example, the ABCD drawers in FIGS. 5A-5B can be applied to any of the aforementioned embodiments. Similarly, the hood in FIGS. 6A-6E could be applied to any of the cart embodiments shown in FIGS. 2-5B, although the hood may have to be oriented to retract side-to-side rather than front-to-back, as appropriate. Moreover, individual hoods could be provided on each of the cardiac and respiratory units of the cart embodiment shown in FIGS. 1A-1H. By way of further example, the exemplary red/blue (cardiac/respiratory) color scheme discussed with respect to some of the cart embodiments, could be applied to any of the embodiments, e.g., by color-coding particular parts of the carts (e.g., the drawers and/or cabinets) and/or ends (or sides) of the carts. In addition, although the rolling members 103 of the carts shown in FIGS. 1A-3 and 5A-6E are shown as being casters, any rolling member may suffice. Moreover, one or more of the rolling members 103 may have fixed axles, similar to the large rolling members 403B of the embodiment shown in FIGS. 4A-4C. Finally, the retractable work surfaces 119 shown with respect to the fourth cart embodiment 400 could also be applied to the other cart embodiments.

Accordingly, these other modifications and variations are fully within the scope of the claimed invention. Therefore, it should be understood that the apparatuses described herein are illustrative only and are not limiting upon the scope of the invention, which is indicated by the following claims.

1. A medical cart comprising:
a cardiac unit comprising:
rolling members that permit the cardiac unit to be rolled to a predetermined location, and
a cardiac storage structure configured to contain at least one cardiac item;
a respiratory unit comprising:
rolling members that permit the respiratory unit to be rolled to the predetermined location, and
a respiratory storage structure configured to contain at least one respiratory item; and
a mechanism configured to releasably join the cardiac unit and the respiratory unit such that the cardiac and respiratory units can be jointly rolled to the predetermined location, and configured to permit the cardiac and respiratory units to be separated at the predetermined location.

2. The medical cart of claim 1, wherein the cardiac storage structure comprises at least one drawer.

3. The medical cart of claim 1, wherein the cardiac unit comprises at least one of a defibrillator platform and an intravenous administration member.

4. The medical cart of claim 1, wherein the respiratory storage structure comprises at least one drawer.

5. The medical cart of claim 1, wherein the respiratory unit comprises a container configured to house an oxygen tank.

6. A medical cart comprising:
a first unit configured to transport at least one item associated with a first medical condition, wherein the first unit has a first visual guide identifying the first unit as transporting items for treating the first medical condition; and
a second unit configured to transport at least one item associated with a second medical condition, the second unit being joined to the first unit, wherein the second unit has a second visual guide identifying the second unit as transporting items for treating the second medical condition.

7. The medical cart of claim 6, wherein the first visual guide is a first color and the second visual guide is a second color.

8. The medical cart of claim 7, wherein the first unit comprises at least one drawer with a drawer face of the first color and being configured to contain at least some of the items associated with the first medical condition, and the second unit comprises at least one drawer with a drawer face of the second color and configured to contain at least some of the items associated with the second medical condition.

9. The medical cart of claim 6, wherein the first unit is a cardiac unit and the first medical condition is a cardiac condition, and the second unit is a respiratory unit and the second medical condition is a respiratory condition.

10. The medical cart of claim 9, wherein the first unit includes at least one of a defibrillator platform and an intravenous administration member and the second unit includes a container configured to house an oxygen tank.

11. A medical cart comprising:
a mobile base;
a plurality of drawers supported by the mobile base and being configured to contain at least one of cardiac and respiratory items, each of the drawers having a front side and a rear side;
at least one cabinet hingedly connected to the mobile base, wherein the cabinet is configured to swing between a closed position, in which the cabinet blocks access to the front sides of the drawers, and an open position, in which the cabinet does not block access to the front side of the drawers.

12. The medical cart of claim 11, wherein the mobile base is configured such that, when the cabinet is in the open position, the drawers can be opened by pulling the front side of the drawer and by pulling the rear side of the drawer, and, when the cabinet is in the closed position, the drawers can be opened only by pulling the rear side of the drawer.

13. The medical cart of claim 11, further comprising:
at least one of a defibrillator platform and an intravenous administration member.

14. The medical cart of claim 11, further comprising:
a container configured to house an oxygen tank.

15. A medical cart comprising:
a plurality of drawers configured to contain items associated with at least one medical condition, wherein each of the drawers has a front side and a rear side; and
a mobile base for supporting the plurality of drawers, wherein the mobile base is configured such that the drawers can be opened by pulling the front side of the drawers and by pulling the rear side of the drawers.

16. The medical cart of claim 15, further comprising:
at least one of a defibrillator platform and an intravenous administration member supported by the mobile base,
wherein the at least one medical condition includes a cardiac condition.

17. The medical cart of claim 15, further comprising:
a container configured to house an oxygen tank,
wherein the at least one medical condition is a respiratory condition.

18. The medical cart of claim 15, wherein a first drawer has a first visual guide identifying the first drawer as containing items for a first medical condition and a second drawer has a second visual guide identifying it as containing items for a second medical condition, and wherein the first and second visual guides are visible on both the front side and the rear side of the first and second drawers.

19. The medical cart of claim 18, wherein the first visual guide is a first color and the first drawer has a drawer face of the first color on the front side and a drawer face of the first color on the rear side, and wherein the second visual guide is a second color and the second drawer has a drawer face of the second color on the front side and a drawer face of the second color on the rear side.

20. A medical cart comprising:
a base comprising rolling members that permit the base to be rolled to a location;
a plurality of drawers configured to contain items associated with at least one medical condition;
a top shelf positioned at least partially over the drawers; and
a retractable hood that is movable between an open position, in which the top shelf is accessible to a user and a closed position, in which access to the top shelf is blocked.

21. The medical cart of claim 20, further comprising:
at least one of a defibrillator platform and an intravenous administration member supported by the base,
wherein that at least one medical condition includes a cardiac condition.

22. The medical cart of claim 20, further comprising:
a container supported by the base and configured to house an oxygen tank,
wherein the at least one medical condition includes a respiratory condition.

23. The medical cart of claim 20, further comprising:
sidewalls supported by the base,
wherein tracks are formed in inner sides of the sidewalls, and wherein edges of the hood are movable in the tracks.

24. A medical cart comprising:
a first unit positioned at a front end of the cart, the first unit including a first storage structure configured to contain at least one item associated with a first medical condition and to permit access to the at least one item associated with the first medical condition from the front end of the cart; and
a second unit positioned at a rear end of the cart, the second unit including a second storage structure configured to transport at least one item associated with a second medical condition and to permit access to the at least one item associated with the second medical condition from the rear end of the cart.

25. The medical cart of claim 24, wherein the first storage structure comprises at least one drawer and the second storage structure comprises at least one drawer.

26. The medical cart of claim 24, further comprising:
at least one of a defibrillator platform and an intravenous administration member supported by the first unit,
wherein the first medical condition is a cardiac condition.

27. The medical cart of claim 24, further comprising:
a container supported by the second unit and configured to house an oxygen tank,
wherein the second medical condition is a respiratory condition.
